(19) United States
(12) Patent Application Publication
(10) Pub. No.: US 2023/0303743 A1
(43) Pub. Date: Sep. 28, 2023

(54) POLYMERIC COATINGS

(71) Applicant: MicroVention, Inc., Aliso Viejo, CA (US)

(72) Inventors: Gregory M. Cruise, Rancho Santa Margarita, CA (US); Xinping Wu, Aliso Viejo, CA (US)

(21) Appl. No.: 18/023,296

(22) PCT Filed: Aug. 31, 2021
(86) PCT No.: PCT/US2021/048464

§ 371 Date: Feb. 24, 2023

Related U.S. Application Data
(60) Provisional application No. 63/072,418, filed on Aug. 31, 2020.

(51) Int. Cl. C08F 220/28; C08L 33/14; A61L 33/06

(52) U.S. Cl. CPC C08F 220/285; C08F 220/281; C08L 33/14; A61L 33/064; A61L 2420/02

(57) ABSTRACT

Described herein are polymeric coatings, methods of preparing the polymeric coatings, methods of using the polymeric coatings, and substrates including one or more surfaces coated with the polymeric coatings.

### RELATED APPLICATIONS

This application is a national stage entry under 35 USC § 371 of Patent Cooperation Treaty application no. PCT/US2021/048464, which claims priority of U.S. Provisional Patent Application No. 63/072,418, filed Aug. 31, 2020, the entire content of each of which is incorporated by reference.

### FIELD

Described herein are polymeric coatings, which can be covalently bound to a substrate. The polymeric coatings may be used to passivate medical devices including luminal devices. The polymeric coatings may impart improved durability or improved thrombogenicity, or both, to the surface on which they are deposited. The Methods of preparing and using the polymers are also described.

### BACKGROUND

Metallic and polymeric substrates are used in a variety of applications, including biomedical applications. In some applications, improved durability of these substrates is desired. In some applications, improved hemocompatibility of these substrates is desired. In some applications, improved durability and hemocompatibility of these substrates is desired. Many formulations have been evaluated as surface coatings. However, there remains a need for satisfactory substrate coatings, including medical device coatings.

### SUMMARY

Described herein are polymers useful as coatings. Also described herein are methods of passivating a surface with a polymeric coating. In some embodiments, the surface is a surface of a substrate. The substrates can be virtually in any form. In some embodiments, the substrate is a medical device. In some embodiments, the substrate is an access device. In some embodiments, the substrate is formed into an implantable medical device. In some embodiments, the substrate may be formed into a stent such as a braided stent platform. In some embodiments, the medical device is a luminal device, such as a luminal delivery device. In some embodiments, the luminal delivery device is a needle, a trocar, a cannula, a stent, or a catheter. In some embodiments, the catheter is a microcatheter. In some embodiments, the medical device is a guide wire, or an implantable medical device such as a stent. Implantable medical devices include, but are not limited to, flat coupons, hypo tubes, wires, woven wires, or laser cut objects. In some embodiments, the medical device is one used to access a lumen of a subject's body.

### DETAILED DESCRIPTION

Described herein are polymeric coatings, which can be covalently bound to a substrate. Also provided herein are metallic or polymeric substrates having its entire surface or a portion of its surface covalently coupled to one or more polymers described herein. In some embodiments, the polymer reduces the thrombogenicity of the substrate, improves the durability of the substrate, or both. In some embodiments the polymer improves the hemocompatibility of the substrate.

### Definitions

Listed below are definitions of various terms used to describe the compositions and methods provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the compositions and methods provided herein pertain. Generally, the nomenclature used herein and the laboratory procedures in polymer chemistry, analytical chemistry, and organic chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed compositions and methods.

### Polymers

In some embodiments, provided herein are co-polymers of:
a) at least one monomer independently selected from alkyloxyalkyl acrylate, alkyloxyalkyl (alkyl)acrylate, heterocycloalkyl acrylate, (heterocycloalkyl)alkyl acrylate, heterocycloalkyl (alkyl)acrylate, or (heterocycloalkyl)alkyl (alkyl)acrylate; and
b) at least one monomer independently selected from a monomer containing an amine, a carboxylic acid, or a hydroxyl, or a salt thereof.

In some embodiments, the co-polymer is a co-polymer of:
a) at least one monomer selected from a monomer of Formula I or a monomer of Formula II; and
b) at least one monomer independently selected from a monomer containing an amine, a carboxylic acid, or a hydroxyl, or a salt thereof; and
wherein
Formula I has the structure

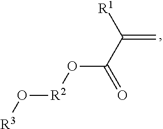

R¹ is H or C₁₋₁₀ alkyl,
R² is C₁₋₁₀ alkylene,
R³ is C₁₋₁₀ alkyl,
Formula II has the structure

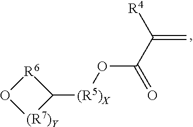

R⁴ is H or C₁₋₁₀ alkyl,
R⁵ is C₁₋₁₀ alkylene,
R⁶ is C₁₋₁₀ alkylene,
R⁷ is C₁₋₁₀ alkylene,
X is 0 or 1, and
Y is 0 or 1.

In some embodiments, R¹ and R⁴ are, independently, H or straight or branched C₁₋₆ alkyl. In some embodiments, R¹ and R⁴ are, independently, H or straight or branched C₁₋₃ alkyl. In some embodiments, R¹ and R⁴ are, independently, H, methyl, or ethyl. In some embodiments, R¹ and R⁴ are, independently, H or methyl. In some embodiments, R¹ is methyl. In some embodiments, R⁴ is methyl. In some embodiments, R¹ is H. In some embodiments, R⁴ is H.

In some embodiments, R³ is straight or branched C₁₋₆ alkyl. In some embodiments, R³ is straight or branched C₁₋₃ alkyl. In some embodiments, R³ is methyl or ethyl. In some embodiments, R³ is methyl.

In some embodiments, R², R⁵, R⁶, and R⁷ are, independently, straight or branched C₁₋₆ alkylene. In some embodiments, R², R⁵, R⁶, and R⁷ are, independently, straight or branched C₂₋₆ alkylene. In some embodiments, R², R⁵, R⁶, and R⁷ are, independently, straight or branched C₁₋₃ alkylene. In some embodiments, R², R⁵, R⁶, and R⁷ are, independently, straight or branched C₂₋₄ alkylene. In some embodiments, R⁵ is methylene.

In some embodiments, X is 1. In some embodiments, X is 0. In some embodiments, Y is 1. In some embodiments, Y is 0. In some embodiments, X is 0 and Y is 0. In some embodiments, X is 1 and Y is 0.

In some embodiments, R⁵ is methylene, X is 1, and Y is 0. In some embodiments, R⁴ is H, R⁵ is methylene, X is 1, and Y is 0.

In some embodiments, R¹ is H and R³ is methyl. In some embodiments, R¹ is H and R³ is ethyl. In some embodiments, R¹ is methyl and R³ is methyl. In some embodiments, R¹ is methyl and R³ is ethyl.

In some embodiments, the co-polymer is a co-polymer of:
a) a monomer of Formula I and a monomer of Formula II; and
b) at least one of a monomer containing an amine, a carboxylic acid, a hydroxyl, or a combination thereof, or a salt thereof.

In some embodiments, the co-polymer is a co-polymer of:
a) a monomer of Formula I or a monomer of Formula II; and
b) a monomer containing an amine, a carboxylic acid, or a hydroxyl, or a salt thereof.

In some embodiments, the co-polymer is a co-polymer of:
a) a monomer of Formula I or a monomer of Formula II; and
b) a monomer containing an amine or a hydroxyl, or a salt thereof.

In some embodiments, the monomer of Formula I is:

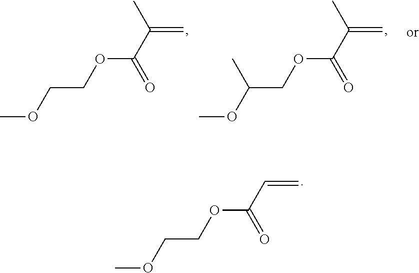

In some embodiments, the monomer of Formula II is:

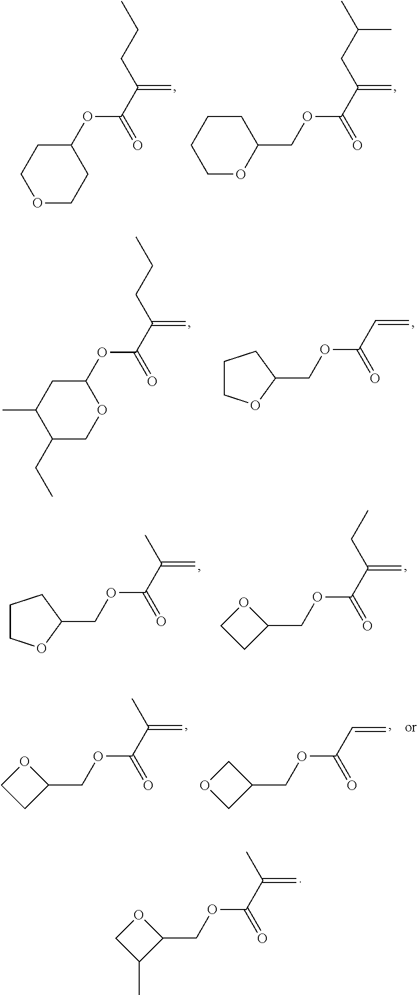

In some embodiments, the co-polymer is a co-polymer of:
a)

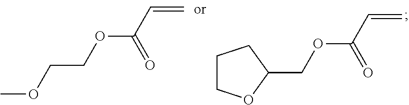

and
b) an aminopropyl methacrylate or an aminopropyl acrylate, or a salt thereof.

In some embodiments, the co-polymer is a co-polymer of:
a)

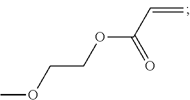

and
b) (3-aminopropyl)methacrylate or a salt thereof.

In some embodiments, the co-polymer is a co-polymer of:
a)

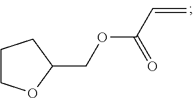

and
b) (3-aminopropyl)methacrylate or a salt thereof.

In some embodiments, the co-polymer is a co-polymer of:
a)

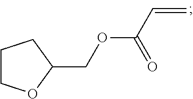

and
b) 4-hydroxybutyl acrylate or a salt thereof.

The polymers described herein are prepared by polymerization of two or more monomers. In some embodiments, the co-polymers described herein are prepared by polymerizing a) at least one monomer independently selected from alkyloxyalkyl acrylate, alkyloxyalkyl (alkyl)acrylate, heterocycloalkyl acrylate, (heterocycloalkyl)alkyl acrylate, heterocycloalkyl (alkyl)acrylate, or (heterocycloalkyl)alkyl (alkyl)acrylate; and b) at least one monomer independently selected from a monomer containing an amine, a carboxylic acid, or a hydroxyl, or a salt thereof. In some embodiments, the polymer is prepared by polymerizing an alkoxyalkyl (alkyl)acrylate or a derivative thereof and a second monomer containing an amine, a carboxylic acid, or a hydroxyl group, or a salt thereof. In some embodiments, the polymer is prepared by polymerizing an alkoxyalkyl acrylate or a derivative thereof and a second monomer containing an amine, a carboxylic acid, or a hydroxyl group. In some embodiments, the polymer is prepared by polymerizing an alkoxyalkyl (meth)acrylate or a derivative thereof and a second monomer containing an amine, a carboxylic acid, or a hydroxyl group.

In some embodiments, the polymer is prepared by polymerizing a heterocycloalkyl acrylate or a derivative thereof and a second monomer containing an amine, a carboxylic acid, or a hydroxyl group. In some embodiments, the polymer is prepared by polymerizing a (heterocycloalkyl)alkyl acrylate or a derivative thereof and a second monomer containing an amine, a carboxylic acid, or a hydroxyl group. In some embodiments, the polymer is prepared by polymerizing a heterocycloalkyl (alkyl)acrylate or a derivative thereof and a second monomer containing an amine, a carboxylic acid, or a hydroxyl group. In some embodiments, the polymer is prepared by polymerizing a (heterocycloalkyl)alkyl (alkyl)acrylate or a derivative thereof and a second monomer containing an amine, a carboxylic acid, or a hydroxyl group. In some embodiments, heterocycloalkyl refers to a cycloalkyl moiety that includes at least one of O, N. or S. In some embodiments, heterocycloalkyl refers to a cycloalkyl moiety where hetero refers to O. In some embodiments, the polymer is prepared by polymerizing tetrahydrofurfuryl acrylate or a derivative thereof and a second monomer containing an amine, a carboxylic acid, or a hydroxyl group.

In some embodiments, the first monomer is selected from alkyloxyalkyl acrylates, alkyloxyalkyl (alkyl)acrylates, heterocycloalkyl acrylates, (heterocycloalkyl)alkyl acrylates, heterocycloalkyl (alkyl)acrylates, or (heterocycloalkyl)alkyl (alkyl)acrylates. In some embodiments, the first monomer is

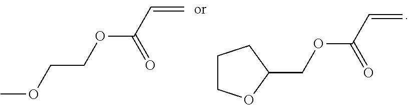

In some embodiments, the second monomer contains a polymerizable moiety as well as an amine, carboxylic acid, or hydroxyl group. In some embodiments, the second monomer contains a polymerizable acrylate or alkylacrylate (for example, methacrylate) as well as an amine, carboxylic acid, or hydroxyl group.

In some embodiments, the second monomer is aminoethyl methacrylate, aminopropyl methacrylamide, a combination thereof, or a derivative thereof. In some embodiments, the second monomer is acrylic acid, methacrylic acid, a combination thereof, or a derivative thereof. In some embodiments, the second monomer is hydroxyethyl methacrylate, hydroxyethyl acrylate, hydropropyl acrylate, hydroxybutyl acrylate, a combination thereof, or a derivative thereof.

Monomers containing amines include 3-aminopropyl methacrylamide, 2-aminoethyl methacrylate, N-(3-methylpyridine)acrylamide, 2-(N,N-dimethylamino)ethyl methacrylate, 2-(N,N-dimethylamino)ethyl acrylate, 2-(tert-butylamino)ethyl methacrylate, methacryloyl-L-lysine, N-(2-(4-aminophenyl)ethyl)acrylamide, N-(4-aminobenzyl)acrylamide, N-(2-(4-imidazolyl)ethyl)acrylamide, a derivative thereof, or a combination thereof.

Monomers containing carboxylic acids include acrylic acid, methacrylic acid, a derivative thereof, or a combination thereof. Monomers containing hydroxyl groups include 2-hydroxyethyl acrylate, 2-hydroxyethyl methacrylate, 2-hydroxypropyl acrylate, 4-hydroxybutyl acrylate, a derivative thereof, or a combination thereof.

Monomers containing hydroxyls include hydroxyethyl methacrylate, hydroxyethyl acrylate, hydropropyl acrylate, hydroxybutyl acrylate, a combination thereof, or a derivative thereof.

To prepare the polymer, the two or more monomers and an initiator are dissolved in a solvent. In general, any solvent that dissolves the two or more monomers and the initiator can be used. Due to the disparate solubility of the alkoxyalkyl (alkyl)acrylate and the monomer containing an amine salt, judicious solvent selection is required.

In some embodiments, the solvents include methanol/water, ethanol/water, isopropanol/water, dioxane/water, tetrahydrofuran/water, dimethylformamide/water, dimethylsulfoxide, water, dimethylsulfoxide/water, or a combination thereof.

With carboxylic acid and hydroxyl containing monomers, a wider range of solvents can be utilized, including toluene, xylene, dimethylsulfoxide, dioxane, THF, methanol, ethanol, dimethyl formamide, or a combination thereof.

Polymerization initiators can be used to start the polymerization of the monomers in the solution. The polymerization can be initiated by reduction-oxidation, radiation, heat, or any other method known in the art. Radiation cross-linking of the monomer solution can be achieved with ultraviolet light or visible light with suitable initiators or ionizing radiation (e.g., electron beam or gamma ray) without initiators. Polymerization can be achieved by application of heat, either by conventionally heating the solution using a heat source such as a heating well, or by application of infrared light to the monomer solution. In some embodiments, the polymerization initiator is azobisisobutyronitrile (AIBN) or a water soluble AIBN derivative such as (2,2′-azobis(2-methylpropionamidine) di hydrochloride or 4,4′-azobis(4-cyanopentanoic acid). Other initiators that can be used include N,N,N′,N′-tetramethylethylenediamine, ammonium persulfate, benzoyl peroxides, or a combination thereof. In some embodiments, the concentrations of the initiator range from 0.25% to 2% w/w of the mass of the monomers in solution. The polymerization reaction is performed at elevated temperatures, preferably in the range from 65 to 85° C. After the polymerization is completed, the polymer is recovered by precipitation in a non-solvent and dried under vacuum.

Substrates suitable for coating with the polymers provided herein may be any suitable metallic or polymeric material. The metallic substrate may be shaped in any convenient geometry, including tubes, rods, sheets, or more complex shapes such as braids or meshworks. In some embodiments, the metallic substrate is a needle, trocar, or cannula. The polymeric substrate may include a shape in any convenient geometry, including tubes, rods, sheets, or more complex shapes. In some embodiments, the polymeric substrate is a film.

In some embodiments, the metallic substrates include stainless steel, cobalt chrome, titanium, or nickel-titanium, alloys thereof, or a combination thereof. In some embodiments, the metallic substrate is stainless steel.

In some embodiments, the polymeric substrates include thermoplastic polyurethanes, thermoplastic elastomers, thermoset elastomers, polyamides, polyesters, polystyrenes, polyether ether ketones, polyethylene vinyl acetates, polyvinylidene fluorides, polypropylenes, polyethylenes, polyvinyl chlorides, polycarbonates, or a combination thereof. In some embodiments, the polymeric substrate is a polyvinyl film.

The polymer can be applied to the substrate in up to four steps, some of which are optional. The necessity of each step can be driven by the selection of the substrate. Step 1 is cleaning. In some embodiments, to clean the substrate, it is incubated in a solvent, such as, acetone, methanol, ethanol, isopropyl alcohol, water, or a combination thereof, under sonication. The duration of each washing step ranges from 1 to 20 minutes. The temperature of sonication ranges from 18 to 55° C. Immediately following the cleaning step, the substrate moves to Step 2.

Step 2 is oxidation, a treatment to increase the number of hydroxyl groups on the surface of the metallic substrate. Metallic and polymeric surfaces may be oxidized using any of a number of different oxidizers, including heat, acids, bases, peroxides, plasma treatment, or a combination thereof. Acids include hydrochloric acid, nitric acid, sulfuric acid, hydrofluoric acid, perchloric acid, or a combination thereof. Bases include sodium hydroxide, ammonium hydroxide, or a combination thereof. Peroxides include hydrogen peroxide, t-butyl peroxide, or a combination thereof. In some embodiments, the oxidizer is hydrogen peroxide. The oxidizer used for hydroxylation may be in a concentration from 1% to 100%. The oxidation duration ranges from 0.25 to 4 hours at temperatures ranging from 18 to 100° C. After oxidation, the substrate may be washed in a solvent, such as acetone, methanol, ethanol, isopropyl alcohol, water, or a combination thereof, with or without sonication. Each wash can range from 1 to 15 minutes in duration. Drying under vacuum may follow washing. In some embodiments, the oxidation utilizes 10% hydrogen peroxide at 100° C. for 45 minutes followed by 5-minute sequential washes in water, ethanol, and acetone followed by drying under vacuum. In some embodiments, the oxidation utilizes “piranha solution,” which is a mixture of sulfuric acid, hydrogen peroxide, and water, followed by 5-minute sequential washes in water, ethanol, and acetone followed by drying under vacuum.

Step 3 is silylation, a treatment to bind to and introduce reactive groups to the substrate. The reactive group of the silane can include acrylate, methacrylate, aldehyde, amine, epoxy, ester, halogen, a combination thereof, or a derivative thereof. The reactive group of the silane must react with the amine, carboxylic acid, or hydroxyl group of the second monomer of the polymer.

With an amine or hydroxyl containing polymer, in some embodiments, the silanes are 3-glycidyloxypropyltrimethoxysilane, 5,6-epoxyhexyltriethoxysilane, 2-(3,4-epoxycyclohexyl)ethyltriethoxysilane, 2-(3,4-epoxycyclohexyl)ethyltri methoxysilane, 2-(3,4-epoxycyclohexyl)ethylmethyldiethoxysilane, (3-glycidoxypropyl)trimethoxysilane, 3-bromopropyltri methoxysilane, 7-bromoheptyltrimethoxysilane, 3-isocyanatopropyltriethoxysilane, 3-isocyanatopropyltrimethoxysilane, 3-chloropropyltriethoxysilane, 3-chloropropyltrimethoxysilane, chlorophenyltriethoxysilane, chloromethyltriethoxysilane, or chloromethyltrimethoxysilane.

With a carboxylic acid containing polymer, in some embodiments, the silanes are 3- aminopropyltrimethoxysilane and/or 1,2-bis(trimethoxysilane)ethane, N-(hydroxyethyl)-N-methylaminopropyl-trimethoxysilane, hydroxymethyltriethoxysilane, [hydroxy(polyethyleneoxy)propyl]-triethoxysilane, (3-glycidoxypropyl)trimethoxysilane, 3-glycidyloxypropyltrimethoxysilane, 5,6-epoxyhexyltriethoxysilane, 2-(3,4-epoxycyclohexyl)ethyltriethoxysilane, 2-(3,4-epoxycyclohexyl)ethyltrimethoxysilane, and 2-(3,4-epoxycyclohexyl)ethylmethyldiethoxysilane.

To perform the silylation, the selected silane must be dissolved in solvent. Suitable solvents include ethanol, methanol, isopropanol, acetic acid, water, isopropanol, butanol, dimethyl formamide, dimethyl sulfoxide, ethyl acetate, toluene, chloroform, dichloromethane, or a combination thereof. In general, any solvent or mixture of solvents may be used that dissolves the silane. The solvents may be present in amounts from 0.1% to about 99.9% by weight. In some embodiments, the solvent percentages range from about 90% to about 99%, most preferably about 97%. The silane may be present in amounts from 0.1% to about 99.9% by weight. In some embodiments, the silane percentages range from about 1% to about 10%, most preferably about 3%. In some embodiments, the silane:solvent system is 94% ethanol, 2% water, 1% acetic acid, and 3% silane.

Following hydroxylation, the substrate may be plasma treated with an argon plasma to clean the surface. In some embodiments, the plasma treating parameters include 365 standard cubic centimeters per minute (sccm) argon flow, 300 watts, and 500 mtorr for 10 minutes. Following plasma treatment, the substrate is placed in the silane:solvent system. The duration of the incubation ranges from 6 to 24 hours at a temperature range from 18 to 55° C. The silylation may be performed with shaking at a rate from about 100 rpm to 250 rpm. In some embodiments, the silylation conditions are incubation for 18 hours at room temperature with shaking at 150 rpm.

After silylation, the substrate may be rinsed in a solvent, such as ethanol, methanol, isopropanol, toluene, water, butanol, dimethyl formamide, dimethyl sulfoxide, ethyl acetate, chloroform, dichloromethane, or a combination thereof. In some embodiments, the rinse is ethanol. The silane layer may then be cured at a temperature ranging from 30 to 150° C. for a duration ranging from 5 to 60 minutes. In some embodiments, the curing conditions are 110° C. for 30 minutes.

Step 4 is polymer coupling, a treatment to covalently couple the polymer to the substrate. During this step, the functional group imparted to the polymer from the second or more monomer is reacted to the functional group imparted to the substrate via the silane. In this step, the polymer is dissolved in a suitable solvent, such as water, buffer, methanol, ethanol, isopropanol, butanol, dimethyl formamide, dimethyl sulfoxide, ethyl acetate, toluene, chloroform, dichloromethane, or a combination thereof. In some embodiments, the solvent is 50% v/v ethanol:50% v/v citric buffer in water pH 7. The concentration of the polymer in the solvent can range from about 0.5% to about 95% in the solvent. In some embodiments, the concentration of the polymer is 1%.

The polymer solution may be applied to the substrate by dip coating, spraying, brushing, or a combination thereof. In one embodiment, the substrate may be immersed in a polymer solution for 1 to 48 hours. In some embodiments, the duration is 18 hours. The incubation may be conducted at temperatures ranging from 18 to 100° C. In some embodiments, the temperature is room temperature. The coupling reaction may be performed with shaking at a rate from about 100 rpm to 250 rpm. In some embodiments, the shaking conditions are 150 rpm.

After incubation, the substrate may be rinsed in a solvent, such as ethanol, methanol, isopropanol, toluene, water, butanol, dimethyl formamide, dimethyl sulfoxide, ethyl acetate, chloroform, dichloromethane, or a combination thereof. In some embodiments, the rinse is 50% v/v ethanol:50% v/v water. After rinsing, the substrate may be dried using heat or vacuum. The substrate may be heated at temperatures ranging from 40 to 100° C., with or without vacuum. In some embodiments, the drying conditions are 40° C. under vacuum. At this point, the substrate may be packaged, for example in a kit that may include instructions for use.

For polymeric surfaces, the silylation step may be eliminated and the surface coated by using the cleaning, oxidation, and coupling steps described herein.

In some embodiments, to clean the substrate, the substrate is incubated in acetone, methanol, ethanol, isopropyl alcohol, water, or a combination thereof under sonication. The duration of each washing step ranges from 1 to 20 minutes. The temperature of sonication ranges from 18 to 55° C.

In some embodiments, oxidation is performed in two steps. Step A is hydrolysis using different oxidizers, including heat, acids, bases, peroxides, or a combination thereof. Acids include hydrochloric acid, nitric acid, sulfuric acid, hydrofluoric acid, perchloric acid, or a combination thereof. Bases include sodium hydroxide, ammonium hydroxide, or a combination thereof. Peroxides include hydrogen peroxide, t-butyl peroxide, or a combination thereof. In some embodiments, the oxidizer for polymeric surfaces is oxygen. After oxidation, the substrate may be washed in acetone, methanol, ethanol, isopropyl alcohol, water, or a combination thereof, with or without sonication. Each wash can range from 1 to 15 minutes in duration. Drying under vacuum may follow washing. Step B is oxidation of the hydroxyl groups imparted to the surface to carbonyl groups. In some embodiments, the route of oxidation is by the use of 2-iodoxybenzoic acid. The duration of Step B ranges from 1 to 24 hours. The temperature ranges from room temperature to 100° C. Alternatively, plasma treatment with oxygen, air, argon, or a combination thereof may be used to oxidize the surface of a substrate. In some embodiments, the polymeric substrate is a polyvinyl chloride (PVC) film substrate. Following the Step B oxidation, chromates are removed via washing with sodium bicarbonate solution and water. The plastic substrate is dried using heat, vacuum, flowing inert gas, or a combination thereof.

For the coupling of a hemocompatible polymer to the polymeric substrate, the functional group, preferably an amine group, imparted to the polymer from the second or more monomer is reacted to the carbonyl group imparted to the substrate to form a Schiff base. Subsequently, the Schiff base is reduced to bind the polymer to the substrate, which may be a PVC film, during which time the film may be shaken for 4-96 hours.

Reducing agents can be sodium cyanoborohydride, sodium borohydride, sodium triacetoxyborohydride, or an equivalent. The reducing agent can be assisted by a catalytic amount of iodine. In some embodiments, the reducing agent combination is sodium borohydride with catalytic amount of iodine. In this step, the polymer is dissolved in water, buffer, methanol, ethanol, isopropanol, butanol, dimethyl formamide, dimethyl sulfoxide, ethyl acetate, toluene, chloroform, dichloromethane, ethylene glycol, dimethoxyethane, or a combination thereof. In some embodiments, the solvent is dimethoxyethane. The concentration of the polymer in the solvent can range from about 0.5% to about 95% by weight in the solvent. In some embodiments, the concentration is 1% by weight.

Also provided herein are compositions comprising the polymers provided herein

### Methods

Also provided herein are methods of using the polymers provided herein. In some embodiments, provided herein are methods of reducing the thrombogenicity of a medical device, comprising coating a surface of the medical device with a polymer provided herein. In some embodiments, provided herein are methods of increasing the durability of a medical device, comprising coating a surface of the medical device with a polymer provided herein. Also provided herein are methods of treating a subject in need thereof, comprising administering a treatment to the subject, wherein the treatment uses a medical device having a polymer disposed on one or more of a surface of the medical device.

### EXAMPLES

### Example 1: Preparation of the Stainless Steel Substrate for Silanization

First, the stainless steel substrate is pre-cleaned using sequential incubations in acetone, ethanol, and water for 5 minutes each while sonicating. The cleaned stainless steel substrate is incubated in a solution of 10% hydrogen peroxide in water for 45 minutes at 100° C. and rinsed three times with water. Again, the stainless steel substrate is cleaned using sequential incubations in acetone, ethanol, and water for 5 minutes each while sonicating. Finally, the stainless steel substrate is dried under vacuum for 18 hours.

### Example 2: Silanization of the Stainless Steel Substrate

The silane solution consisting of 94% ethanol, 3% 7-bromoheptyltrimethoxy silane, 2% water, and 1% acetic acid is prepared and allowed to pre-react for 60 minutes. During the pre-reaction period, the stainless steel substrate from Example 1 is plasma treated with an argon plasma (365 seem Ar, 300 watts, 500 mtorr) for 10 minutes.

Subsequently, the stainless steel substrate is immersed in the silane solution and incubated for 18 hours at room temperature with orbital shaking at 150 revolutions per minute while protected from light. At the conclusion of the incubation, the stainless steel substrate is rinsed with ethanol and cured at 110° C. for 30 minutes.

### Example 3: Silanization of the Stainless Steel Substrate

A mixture of silane is made using 7-bromoheptyltrimethoxysilane and 1,2-bis(trimethoxysilane)ethane at a ratio of 9:1 (v/v). The silane mixture is diluted by toluene to 5% by volume and allowed to pre-react for about 60 minutes. During this time, the stainless steel substrate from Example 1 is plasma treated according to the parameters described in Example 2. Subsequently, the stainless steel substrate is immersed in the silane solution and incubated for 18 hours at 70° C. At the conclusion of the incubation, the stainless steel substrate is rinsed with toluene and cured at 110° C. for 30 minutes.

### Example 4: Preparation of Copolymer of Poly(2-Methoxyethyl Acrylate)-co-Poly[(3-Aminopropyl) Methacrylate Hydrochloride]

To a mixture of 40 mL water and 40 mL methanol, 40 g of 2-methoxyethylacrylate, 4 grams of 3-aminopropyl methacrylate hydrochloride, and 440 mg of [4,4′-azobis(4-cyanovaleric acid)] are dissolved. Polymerization occurs over 4 hours at 80° C. The copolymer is recovered by precipitation in a mixture of isopropanol:hexanes (500 mL:500 mL). The copolymer is re-dissolved in a mixture of 80 mL tetrahydrofuran and 20 mL ethanol and re-precipitated in a mixture of isopropanol:hexanes (400 mL:600 mL). The copolymer is re-dissolved in a mixture of 80 mL tetrahydrofuran and 20 mL ethanol and re-precipitated in a mixture of isopropanol:hexanes (300 mL:700 mL). Finally, the copolymer is stirred in 1 L of hexane for 1 hour and dried under vacuum. The copolymer is a white, foamy solid.

### Example 5: Preparation of Copolymer of Poly(Tetrahydrofuryl Acrylate)-co-Poly[(3-Aminopropyl) Methacrylate Hydrochloride]

To a mixture of 40 mL water and 40 mL methanol, 40 g of tetrahydrofurfuryl acrylate, 4 grams of 3-aminopropyl methacrylate hydrochloride, and 440 mg of [4,4′-azobis(4-cyanovaleric acid)] are dissolved. Polymerization occurs over 20 hours at 65° C. The copolymer is recovered by precipitation in a mixture of isopropanol/hexanes (500 mL:500 mL). The copolymer is re-dissolved in 100 mL tetrahydrofuran and re-precipitated in a mixture of isopropanol:hexanes (400 mL:600 mL). The copolymer is re-dissolved in 100 mL tetrahydrofuran and re-precipitated in a mixture of isopropanol:hexanes (300 mL:700 mL). Finally, the copolymer is stirred in 1 L of hexane for 1 hour and dried under vacuum. The copolymer is a slightly orange, foamy solid.

### Example 6: Preparation of the Stainless Steel Substrate Using Copolymer of Poly(2-Methoxyethyl Acrylate)-co-Poly[(3-Aminopropyl)Methacrylamide Hydrochloride)

The copolymer of Example 4 is dissolved in 50%/50% of ethanol/citric buffer 7.0 pH (v/v) at a final concentration of 10 mg/mL. The stainless steel substrate of Example 2 is placed into a vial containing the copolymer solution and incubated for 18 hours at room temperature on the orbital shaker at 150 rpm. After incubation, the device is rinsed with 50%/50% ethanol/water and cured at 40° C. for 30 minutes under vacuum.

### Example 7: Preparation of a Polyvinyl Chloride Film Substrate

The polyvinyl chloride (PVC) substrate is plasma treated with an oxygen plasma for 100 sec. using the conditions described in M. Ghoranneviss, S. Shahidi and J. Wiener, Surface modification of poly vinyl chloride (PVC) using low pressure argon and oxygen plasma, Plasma Science and Technology 12 (2010) 204-207.

### Example 8: Oxidation of a PVC Film Substrate

The PVC substrate from Example 7 is immersed in a solution of 2-iodobenzoic acid in dimethyl sulfoxide (DMSO). The PVC substrate is shaken in this solution for 12 hours at room temperature. Subsequently, the PVC substrate is removed from DMSO solution and rinsed with saturated sodium bicarbonate solution and distilled H₂0 sequentially. The film is dried at ambient conditions.

### Example 9: Coupling of Copolymer of Poly(2-Methoxyethyl Acrylate)-co-Poly[(3-Aminopropyl) Methacrylate Hydrochloride] to the PVC Film Substrate

A solution of poly(2-methoxyethyl acrylate)-co-poly[(3-aminopropyl) methacrylate hydrochloride] in ethanol is mixed with dimethoxy ethane. The ethanol in this solution is then evaporated on a rotary evaporator to leave behind only the polymer and dimethoxy ethane. The final concentration of the polymer in the dimethoxy ethane solution is adjusted to be 1%. The PVC substrate is shaken in this solution overnight. Sodium borohydride is added to the solution and the film is shaken in the solution for 4 hours. If needed, a catalytic amount of iodine can be added to the solution to accelerate the reduction. Upon completion, the PVC film substrate is lifted out of the solution and rinsed three times with distilled H20. The film is dried at ambient conditions.

### Example 10: Preparation of a Copolymer of Tetrahydrofurfuryl Acrylate and 4-Hydroxybutyl Acrylate

To a 2 liter flask under inert atmosphere is added tetrahydrofurfuryl acrylate (100 mL) 4-hydroxybutyl acrylate (24 mL), and toluene (330 mL). Heat the reaction to 75° C. In a separate flask, dissolve 1.2 g of AIBN in 35 mL of Toluene. Add the AIBN solution to the reaction flask, and let the reaction stir overnight at 75° C. At the end of the reaction, remove the heating source and let the reaction cool to room temperature. Pour the content of the flask over 1200 mL of MTBE. Precipitate the polymer by stirring for 20 min. Decant the liquid and keep the solid. Pour 350 mL of MTBE over the solid and stir for 10 min. Decant the liquid and keep the solid. Dissolve the solid in 150 mL of acetone. Pour the polymer solution over 1,300 mL of MTBE and stir for 20 min. Decant the liquid and keep the solid. Dry the solid under reduced pressure.

### Example 11: Preparation of a Stainless Steel Surface or PVC Surface for Coupling

Dissolve the polymer in Example 10 in Dowanol PMA or methanol to afford a 1% solution. Dip the stainless steel or PVC substrate in the polymer solution and let it air dry. The substrates now are ready for coating with copolymers from Examples 4, 5, and 6.

### Example 12: Coating a Stainless Steel Surface or PVC Surface with Copolymers

Dissolve the copolymers obtained from Examples 4, 5, or 6 in ethanol to afford a 1% solution. Dip the stainless steel or PVC substrates in the copolymer solution and let it air dry.

### Incorporation by Reference and Equivalents

The contents of all references (including literature references, issued patents, published patent applications, and co-pending patent applications) cited throughout this application are hereby expressly incorporated herein by reference in their entireties.

Those skilled in the art will recognize, or be able to ascertain using no more than routine experimentation, numerous equivalents to the specific procedures, embodiments, claims, and examples described herein. Such equivalents are intended to be encompassed by the following claims.

It is to be understood that wherever values and ranges are provided herein, all values and ranges encompassed by these values and ranges, or a combination of these values and ranges, are meant to be encompassed within the scope of the embodiments provided herein. Moreover, all values that fall within these ranges, as well as the upper or lower limits of a range of values, are also contemplated by the present application.

We claim:

1. A co-polymer of:
a) at least one monomer independently selected from alkyloxyalkyl acrylate, alkyloxyalkyl (alkyl)acrylate, heterocycloalkyl acrylate, (heterocycloalkyl)alkyl acrylate, heterocycloalkyl (alkyl)acrylate, or (heterocycloalkyl)alkyl (alkyl)acrylate; and
b) at least one monomer independently selected from a monomer containing an amine, a carboxylic acid, or a hydroxyl, or a salt thereof.

2. The co-polymer of claim 1, being a co-polymer of:
a) at least one monomer selected from a monomer of Formula I or a monomer of Formula II,
wherein
Formula I has the structure 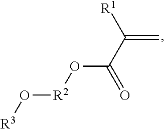
R¹ is H or C₁₋₁₀ alkyl,
R² is C₁₋₁₀ alkylene,
R³ is C₁₋₁₀ alkyl,
Formula II has the structure 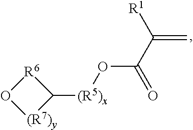
R⁴ is H or C₁₋₁₀ alkyl,
R⁵ is C₁₋₁₀ alkylene,
R⁶ is C₁₋₁₀ alkylene,
R⁷ is C₁₋₁₀ alkylene,
X is 0 or 1, and
Y is 0 or 1; and
b) at least one monomer independently selected from a monomer containing an amine, a carboxylic acid, or a hydroxyl, or a salt thereof.

3. The co-polymer of claim 2, being a co-polymer of:
a) a monomer of Formula I and a monomer of Formula II; and
b) at least one of a monomer containing an amine, a carboxylic acid, a hydroxyl, or a combination thereof, or a salt thereof.

4. The co-polymer of claim 2, being a co-polymer of:
a) a monomer of Formula I or a monomer of Formula II; and
b) a monomer containing an amine, a carboxylic acid, or a hydroxyl, or a salt thereof.

5. The co-polymer of claim 2, being a co-polymer of:
a) a monomer of Formula I or a monomer of Formula II; and
b) a monomer containing an amine or a hydroxyl, or a salt thereof.

6. The co-polymer of one of claims 1-5, wherein the monomer of Formula I is:

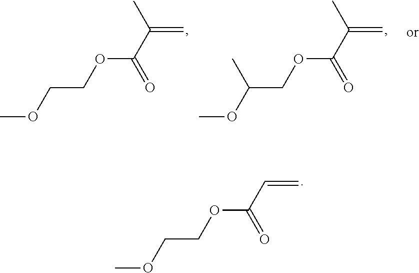

7. The co-polymer of one of claims 1-6, wherein the monomer of Formula II is:

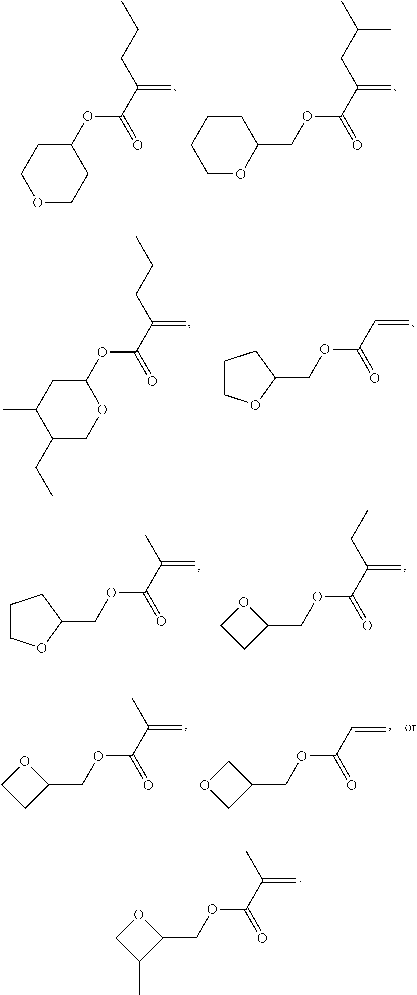

8. The co-polymer of claim 2, being a co-polymer of:
a) 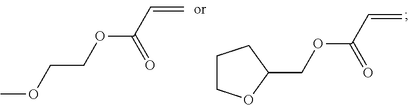
and
b) an aminopropyl methacrylate or an aminopropyl acrylate, or a salt thereof.

9. The co-polymer of claim 2, being a co-polymer of:
a) 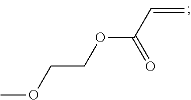
and
b) (3-aminopropyl)methacrylate or a salt thereof.

10. The co-polymer of claim 2, being a co-polymer of:
a) 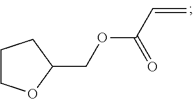
and
b) (3-aminopropyl)methacrylate or a salt thereof.

11. The co-polymer of claim 2, being a co-polymer of:
a) 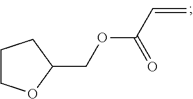
and
b) 4-hydroxybutyl acrylate or a salt thereof.

12. A substrate, including a surface coated with the co-polymer of one of claims 1-11.

13. The substrate of claim 12, wherein the surface is a metallic or polymeric surface.

14. The substrate of claim 12, wherein the surface includes stainless steel, cobalt chrome, titanium, nickel-titanium, an alloy thereof, or a combination thereof.

15. The substrate of claim 12, wherein the surface is the surface of a needle, trocar, or cannula.

16. The substrate of claim 12, wherein the surface includes a thermoplastic polyurethane, thermoplastic elastomer, thermoset elastomer, polyamide, polyester, polystyrene, polyether ether ketone, polyethylene vinyl acetate, polyvinylidene fluoride, polypropylene, polyethylene, polyvinyl chloride, polycarbonate, or a combination thereof.

17. The substrate of one of claims 12-16, wherein the substrate is a medical device.

18. A method of reducing the thrombogenicity of a medical device, comprising coating a surface of the medical device with the co-polymer of one of claims 1-11.

19. A method of increasing the durability of a medical device, comprising coating a surface of the medical device with the co-polymer of one of claims 1-11.

20. A method of treating a subject in need thereof, comprising administering a treatment to the subject, wherein the treatment uses a medical device having the co-polymer of one of claims 1-11 disposed on one or more surfaces of the medical device.

21. A composition, comprising the co-polymer of one of claims 1-11.

22. An apparatus, comprising the co-polymer of one of claims 1-11 disposed on surface of the apparatus.

23. A kit, comprising the substrate of claim 17, and instructions for use thereof.
